(19) United States
(12) Patent Application Publication
(10) Pub. No.: US 2008/0146487 A1
(43) Pub. Date: Jun. 19, 2008

(54) Multiphase bathing tablets

(76) Inventors: Amanda L. O'Connor, Appleton, WI (US); Marcy E. Sojka, Neenah, WI (US); Frank G. Druecke, Oshkosh, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/638,938

(22) Filed: Dec. 13, 2006

(51) Int. Cl. A61L 9/04

(52) U.S. Cl. 512/4

(57) ABSTRACT

Bathing tablets having multiple layers to create different phases in the user experience. As a layer melts away in the shower or bath tub, a new layer having at least one different property lets the user know that a change in the bathing tablet has occurred. Different properties include visual, aromatherapy, effervescent, and exothermic.

### BACKGROUND OF THE INVENTION

The present invention relates generally to bathing preparations, and more particularly to dissolvable tablets that may be used in the bath tub and shower.

Bath and bathing therapies have been known for centuries. As early as the times of ancient Egypt, wealthy families availed themselves of “scented and anointed waters” to allegedly alleviate illness, or to perfume the bath and body.

Some modern bath therapies involve the use of commercially available compounds to create tablets that may be effervescent, perfumed, and/or colored. When such tablets are dropped in a tub of water, bubbling action is noticeable, and the water becomes perfumed and possibly tinted in color. Other modern bath therapies include scented tablets for use in a shower to release vapors that are therapeutic. However, it is well known that the human olfactory senses are not able to detect most odors after a certain period of exposure to the odor. For instance, if one walks into a room that has been scented by aromatic compounds, it becomes immediately noticeable. After a few minutes, the scent is far less noticeable, and any initial pleasure the new scent evoked has dissipated. Therefore, the use of bathing preparations, while initially pleasing, may become far less exciting to the user well before the bathing session is over. Further, the user may not realize that the product provides an advantage throughout the entire bathing session.

Neither the historically-known baths, nor the more modern compositions disclose any bath therapy that provides a cue that the product is working after a period of exposure.

Therefore, there remains a need to provide a bathing preparation that provides an additional level of noticeability to the user after a period of exposure.

### SUMMARY OF THE INVENTION

In one aspect of the invention, a bathing tablet is adapted to outwardly exhibit a property change to a user as it dissolves. The bathing tablet includes a first layer having a second layer disposed on the first layer. The first layer exhibits a different effervescent or exothermic property than the second layer.

In another aspect of the invention is a method of manufacturing a multi-layer bathing tablet that will outwardly exhibit a property change to a user as it dissolves. The method includes the steps of preparing an effervescent first compound having a binder and a first fragrance, and preparing a second compound having a binder and a second fragrance. The first compound is layered on the second compound to create a first layer and a second layer. The first layer and the second layer are pressed together to form a tablet.

In yet another aspect of the invention is a bathing tablet adapted to outwardly exhibit a property change to a user as it dissolves. The bathing tablet includes a first layer having a binder, a first percentage by weight of an acid, a first percentage by weight of a carbonate salt, and a first fragrance. A second layer is disposed on the first layer; the second layer having a binder, a second percentage by weight of an acid, a second percentage by weight of a carbonate salt, and a second fragrance.

### BRIEF DESCRIPTION OF THE DRAWINGS

FIG. 1 is a perspective view of a first embodiment of the present invention.

FIG. 2 is a side elevation of a second embodiment of the present invention.

FIG. 2A is a cross-sectional plan view of the embodiment shown in FIG. 2, taken along the plane defined by line 2a-2a in FIG. 2.

FIG. 3 is a partial cross-sectional side view of a third embodiment of the present invention.

FIG. 3A is a cross-sectional partial plan view of the embodiment shown in FIG. 3, taken along the planes defined by line 3a-3a in FIG. 3.

### DETAILED DESCRIPTION OF THE INVENTION

The present invention is a bathing tablet that has multiple layers to effect phases of feedback to the user. The feedback serves as a cue or indicator that the state of the tablet has undergone a change during a user's bathing or personal spa routine. The feedback may be directed to one or more of the senses, including hearing, sight, smell, or feel. The feedback may be a change in dissolution rate, a change in effervescence, a change in temperature through exothermic reaction, a change in color or other aesthetic effect, a change in fragrance, or a combination thereof. Thus, the feedback is caused by a change in one or more properties that the bathing tablet has at any given time during its intended use.

Some exemplary embodiments of the present invention are presented. The various tablet structures of the present invention are initially disclosed, followed by a discussion of the properties that might be present within each layer of the tablet structure. General and specific examples of tablets in accordance with the present invention are disclosed thereafter. Despite the fact that various specific compositions of bathing tablets for use in the bath and shower are set forth below, it is envisioned that alternate compositions of the present invention may be adopted without deviating from the invention.

### Tablet Structure

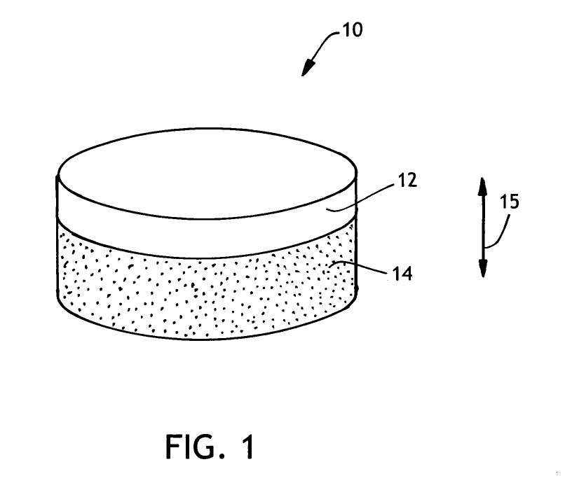

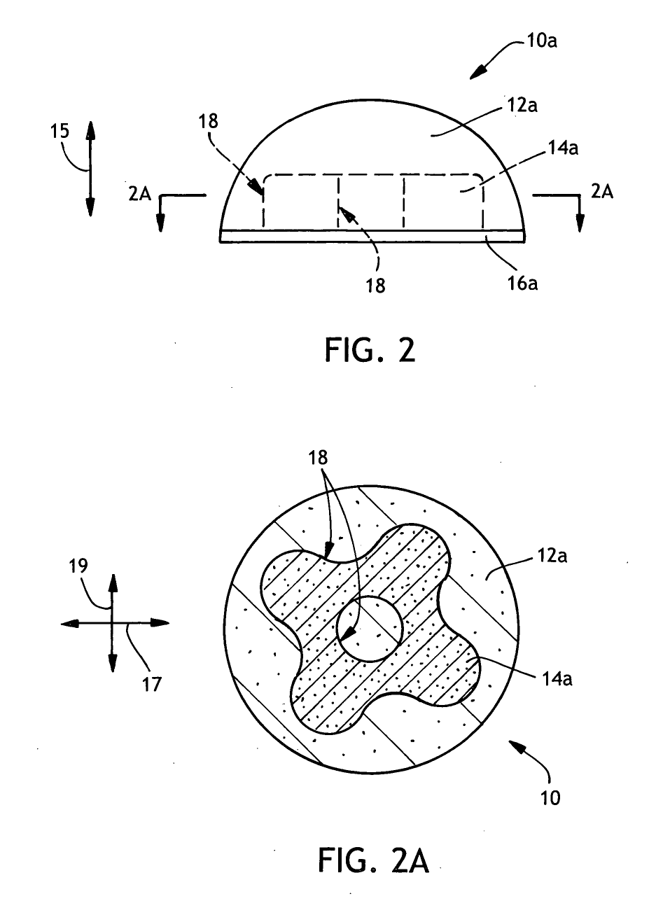

Referring to FIGS. 1 and 2, an embodiment of a bathing tablet 10 of the present invention includes different layers of material in a stacked, cylindrical configuration. Each layer of material has at least one property or characteristic that is different than another layer in direct contact therewith. The bathing tablet of FIG. 1 includes two layers, a first layer 12 and a second layer 14. It is further contemplated that tablet 10 may have more than two layers. For instance, tablet 10 may have three, four, five, six, or more layers. Each layer is about equal in diameter and aligned to form a cylindrical shape. Each layer may have the same or a different thickness as measured in the z-direction 15.

The tablet 10, as shown in FIG. 1, may be most suitable for use in the shower because the streaming water from the shower will contact one of the layers more than the other layers. For instance, if layer 12 is in an upward position during use so that layer 14 makes contact with the shower floor, layer 12 will be exposed to a higher flow rate of water and/or a higher water pressure than layer 14. Thus, if layers 12 and 14 have equivalent rates of dissolution, then layer 12 would dissolve before layer 14, leaving layer 14 exposed to the stream of water from the shower and in full view of the user.

The tablet 10 may have a shape other than a cylinder. It is contemplated that tablet 10 could have layers forming other geometric shapes such as a rectangle, triangle, parallelogram, or oval, or may have a holiday-specific shape such as a gingerbread man, a bell, a tree, an egg, a star, and the like. The precise shape of tablet 10 is not meant to limit the scope of the present invention.

In addition, in one embodiment of the present invention, one of the layers, such as layer 14, may be made from material that is not soluble in water, such as a plastic material. This “dummy” layer may have various colors, textures, or scents. The dummy layer may be useful as a base layer for placing against the shower floor or the like, in order to elevate the remaining layer or layers above the shower floor. Further, the dummy layer may have indicia printed thereon or indicia created by a molding process. The indicia may include instructions regarding use of the tablet. In the alternative, the indicia may indicate that the dummy layer is a coupon good for future purchase. The indicia may also be a pleasant phrase, an aesthetic design, or trivia. It is further contemplated that the indicia may be a proof of purchase. Because the dummy layer is has two opposite surfaces, indicia may be included on both surfaces, yet may serve different purposes, e.g. instructions may be placed on the outer surface, and a coupon be placed on an inner surface facing the remaining layers. The coupon surface would not be visible until the other layer(s) have been dissolved. It is even further contemplated that the dummy layer may be scented and reusable as a sachet. Other layers, such as layer 12, may be attached to the dummy layer by an adhesive or by a mechanical method, e.g. a texture that would engage the surface of the dummy layer that faces layer 12.

Referring to FIGS. 2 and 2A, another embodiment of a bathing tablet, designated as tablet 10a, may have three layers or more. Bathing tablet 10a may be similar in shape to a truncated sphere or dome shape. The shape is defined by a top layer 12a and a base layer 16a. The base layer 16a is relatively flat with the intent that a user will place the base layer against the floor during use. A hidden layer 14a has sides 18 rising in the z-direction 15 that are at least partially surrounded by the top layer 12a as shown, or the base layer 16a (not shown). It is contemplated that sides 18 could be partially surrounded by both layers 12a and 14a (also not shown). Desirably, layer the hidden layer 14a and top layer 16a are disposed against the base layer 16a. Bathing tablet 10a may be suitable for use in the bathtub or shower because the hidden layer 14a is revealed after the top layer 12a and/or base layer 16a have dissolved.

Referring to FIG. 2A, hidden layer 14a may have an aesthetic shape, such as the hollow flower-shape, shown by way of example. The aesthetic shape is largely determined by the area between sides 18 when viewed in a plane oriented in x-direction 17 and y-direction 19.

Hidden layer 14a may have any geometric shape, such as a square, parallelogram, circle, rectangle, triangle; any organic shape such as a shell, flower, plant; a logo such as a trademark, and the like. Likewise, the layers 12a and 16a are not limited to the formation of a dome shape. Any novelty or geometric shape could be used, e.g. an architectural shape, a plant or flower shape, an organic shape such as a shell, a holiday shape such as a star or bell, and the like. The shapes set forth in the example of FIGS. 2 and 2A is not meant to limit the scope of the invention.

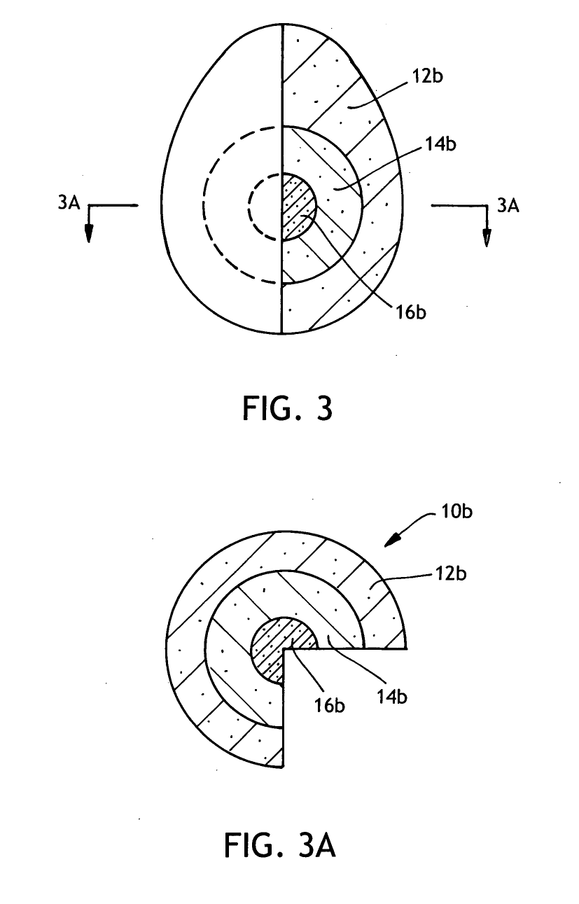

Shown in FIG. 3 is another embodiment of the present invention, bathing tablet 10b. Bathing tablet 10b may have an egg shape outer layer 12b, or a substantially spherical shaped outer layer 12b (not shown). Bathing tablet 10b is shown as having two substantially spherical inner layers, an inner layer 14b and a core 16b. It is contemplated that there may be only one inner layer or more than two inner layers. It is further contemplated that the layer 14b could be omitted to leave a hollow region in which the core 16b could move about.

Unless a hollow region is desired within a tablet, in all three of the examples shown in FIGS. 1-3, if two, three, or more layers are present, it is most suitable that each layer have very little or substantially zero gapping between the layers.

### Properties

Referring now to Table 1, it is noted that the bathing tablets 10-10b of the present invention may be more effectively used in a shower or bath environment depending on which properties the tablet has, or which sense is being stimulated. For example, tablet layers having an effervescent property may be most suitable for use in the shower if the effervescent property is used to stimulate the user's auditory, visual, or tactile senses. It may be possible to hear, see, or feel the bubbles caused when the effervescent layer contacts water.

In contrast, it is difficult to smell bubbles unless the layer also has an aromatherapy property as described herein. The term “aromatherapy” as used in the context of this invention means that the tablet of the present invention emanates an aroma to enhance a feeling of well-being or health. If an aromatherapy property is desired, then it is likely used only to stimulate the olfactory senses because scents cannot be seen, touched, or heard.

The term “exothermic” as used in the context of this invention means that the layer may produce enough heat to cause vapor to emanate from the layer. For instance, water from a hot shower may contact the tablet containing, for example, MgCl₂. The reaction between a compound like MgCl₂ and water will cause warm water to further increase in temperature, and may create a small amount of water vapor. If a layer exhibits an exothermic property, then the visual (e.g. vapor) or tactile senses (e.g. heat) may be stimulated in the shower. However, it may be difficult to feel an exothermic reaction in the bathtub because the mass of water may be too great for a significant heat to be maintained for a visual or tactile effect.

Finally, if a layer exhibits a visual property, such as a particular color, design, or aesthetic feature, the only sense that will be stimulated in the shower or bath is the visual sense.

TABLE 1
Tablet properties vs. ability to affect
user senses in the bath or shower.

| | | | Visual | |
|---|---|---|---|---|
| | | | (color or | |
| Sense | Effervescent | Exothermic | design) | Aromatherapy |
| Auditory | Shower | | | |
| Olfactory | | | | Shower/Bath |
| Visual | Shower/Bath | Shower | Shower/ | |
| | | | Bath | |
| Tactile | Shower/Bath | Shower/ | | |
| | | Bath | | |

Layers that define the tablets 10-10b may have one or more of the properties listed in Table 1. The following examples are representative of the many combinations of properties or sensory effects a tablet may have. Regardless of the components delivering a certain property, each tablet layer may further include a binder selected from the list of sorbitol, or xylitol, or malto-dextrin, or lactose, or other known binders for tablet pressing.

Effervescent Property:

Effervescent compositions combine carbonate salts such as sodium carbonate and/or sodium bicarbonate with acidic materials such as citric, malic, or fumaric acid in a way that carbon dioxide gas is generated by the reaction that occurs when the acid and carbonate come into intimate contact with each other. Naturally, situations that allow the materials to dissolve in close proximity to each other accelerate the reaction.

For example, the reaction between one acid and carbonate salt, e.g. citric acid and sodium bicarbonate, is as follows:

(one)C6H8O7+(three)NaHCO3+H2O→(four)H2O+(three)CO2+(one)Na3C6H5O7

Since the presence of water can initiate the effervescent reaction, these products are suitably formulated, manufactured, and packaged in ways that prevent unintended contact with moisture. Alternate compositions are disclosed in U.S. Pat. No. 5,993,854, issued to Needleman et al. on Nov. 30, 1999, incorporated by reference to the extent it is consistent with the present invention.

### Exothermic Property:

Various compositions are known which have exothermic heats of solution. The greater the positive value of the heat of solution, the more heat is liberated per gram-mole of the substance. Thus, materials with higher heats of solution are able to raise the temperature of a given amount of water higher than compounds with lower heats of solution. A useful example of a common chemical compound with a high exothermic heat of solution for possible use in a dissolving tablet is magnesium chloride. It is known that when one mole of magnesium chloride is dissolved in an adequate amount of water, the amount of heat generated is about 35.9 kilocalories. Magnesium chloride is a naturally occurring compound and is one example of a chemical compound for possible inclusion in a dissolving tablet, and more specifically in a layer or layers of a multiphase bathing tablet. Another example of a chemical compound with a high exothermic heat of solution for possible use in a dissolving tablet is magnesium sulfate, in a non-hydrated form. A number of compositions having exothermic heats of solution are listed in the following references: U.S. Pat. No. 5,993,854 issued to Needleman et al. on Nov. 30, 1999; Dept. of the Army, U.S. Army Soldier and Biological Chemical Command Natick Solder Center, Report NATICK/TR-01/004, Applications of New Chemical Heat Sources Phase 1 (January 2001), both incorporated by reference to the extent they are consistent with the present invention.

### Visual Property:

Various dyes may be added to a layer to create a desired color. In addition, the layer may be shaped into an aesthetic or even functional design, as described above. A tablet or layer surface may be printed or embossed with an indicia such as a trademark or design. Moreover, a layer surface that is only exposed after being exposed to water may be printed or stamped with an indicia. Thus, the layers may have a texture that only becomes apparent after a covering layer has at least partially dissolved.

### Aromatherapy Property:

There have also been many studies conducted which have speculated that aroma greatly influences human psychology and physiology, and the term “aromatherapy” has been used to describe the beneficial properties which can be achieved using fragrances. U.S. Pat. No. 5,238,915, incorporated herein to the extent it does not conflict with the present invention, discloses various aromatic compositions and perfumes discussed in relation to aromatherapy. Prolonged release of perfume may be accomplished by a pH adjusting tablet to provide a variable release of the perfume.

Other additives to the layers of tablet 10-10b may include: a moisturizer, vitamins, effective ingredients of spring, protease, lanoline, silicone, crude drug or extract thereof, etc.

### GENERAL EXAMPLES

### Example 1

Tablet 10 has a first layer 12 with an aromatherapy property that is different than second layer 14. Optionally, one of the layers 12 or 14 exhibits an exothermic property. Further optionally, one of the layers, 12 or 14 is a different color. Moreover, one of the layers may have an effervescent property. Each layer may have the same dissolution rate.

### Example 2

Tablet 10a has a first layer 12a with a color that is different than second layer 14a. Layer 16a may be identical to layer 12a with respect to color. In an alternative embodiment, layer 16a may be different in color that both layers 12a and 14a.

Suitably, layer 14a has a lower dissolution rate than both the layer 12a and layer 16a. In the alternative, layer 14a has a lower dissolution rate than layer 16a, but a higher dissolution rate than layer 12a. In yet another alternative, layer 14a has a higher dissolution rate than both layers 12a and 16a; and layer 16a has a lower dissolution rate than layer 12a.

In one embodiment, layer 14a has an effervescent property and the remaining layers are non-effervescent. In an alternative embodiment, the layer 12a and/or layer 16a has an effervescent property and the remaining layer 14a does not. In another embodiment, the layer 12a has an exothermic property.

### Example 3

Tablet 10b has a first layer 12b with an aromatherapy property that is different than second layer 14b and core 16b. Tablet 10b layer 14b may have an aromatherapy property that is different than core 16b. Either the layer 14b is effervescent, or the layer 12b and core 16 are effervescent. The layers may have different dissolution rates. Optionally, each layer is has a color that is different than a neighboring layer. For instance, layer 12b is different in color than layer 14b, and layer 14b is different in color than core 16b.

The rate at which the tablets 10-10b go through a change in the dominant property is dependent upon the dissolution rate of each layer forming a tablet, 10-10b. The dissolution rate is the rate at which a tablet layer dissolves when placed in water, as described by the test method herein.

### SPECIFIC EXAMPLES

### Example 4

One example of a tablet 10 comprises two layers 12 and 14, with each layer having a different composition. The composition of the layer 12 of this example may be a mixture of four components:
1. a binder such as malto-dextrin as about 45% of the total batch weight;
2. citric acid as about 25 to about 27% of the total batch weight;
3. sodium carbonate as about 25 to about 27% of the total batch weight; and
4. liquid fragrance; e.g. menthol or eucalyptus, as about 1% to about 5% of the total batch weight.

The four components may be dry blended together and then used as feedstock to make layer 12 in a two-layer tablet press as is known in the art. Suitable tablet pressing equipment is available from companies such as Fette, or Korsch. The appearance of layer 12 of the tablet is a color such as white (or whatever the color the ingredients are without additional dyes). Also, layer 12 may effervesce as water dissolves the citric acid and sodium carbonate components. Further, layer 12 may liberate a volatile organic fragrance as the menthol or eucalyptus component is warmed and released into the air.

The composition of the layer 14 may include the following five components:
1. a binder such as malto-dextrin as approximately 45% of the total batch weight;
2. citric acid as approximately 25 to 27% of the total batch weight;
3. sodium carbonate as approximately 25 to 27% of the total batch weight;
4. liquid fragrance; i.e. lavender, as approximately 1% to 4% of the total batch weight; and
5. dye or food coloring; e.g blue, as approximately less than 1% of the total batch weight.

The five components may be dry blended together and as feedstock for layer 14 in a two-layer tablet press as is known in the art. The appearance of layer 14 may be a color other than white, such as blue. Also, layer 14 may generate effervescence as water dissolves the citric acid and sodium carbonate components. Like layer 12, layer 14 may liberate a volatile organic fragrance as the lavender component is warmed and released into the air. The bottom layer will appear blue.

Thus, if tablet 10 is placed in a shower under a stream of water with layer 12 in an upward position and layer 14 contacting the shower floor, the appearance of tablet 10 will change from white to blue as layer 12 is dissolved away. Further, the scent given off by tablet 10 will change from a predominantly eucalyptus or menthol, to a lavender scent.

### Example 5

One example of a tablet 10 comprises three layers, with each layer having a different composition. The composition of the first layer of this example may be a mixture of four components:
1. a binder such as malto-dextrin as about 45% of the total batch weight;
2. citric acid as about 25 to about 27% of the total batch weight;
3. sodium carbonate as about 25 to about 27% of the total batch weight; and
4. liquid fragrance; e.g. menthol or eucalyptus, as about 1% to about 5% of the total batch weight.

The four components may be dry blended together and then used as feedstock to make the first layer in a tablet press as is known in the art. The appearance of the first layer of the tablet is a color such as white (or whatever the color the ingredients are without additional dyes). Also, the first layer may effervesce as water dissolves the citric acid and sodium carbonate components. Further, the first layer may liberate a volatile organic fragrance as the menthol or eucalyptus component is warmed and released into the air.

The composition of the second layer may include the following five components:
1. a binder such as malto-dextrin as approximately 25% of the total batch weight;
2. citric acid as approximately 35 to 36% of the total batch weight;
3. sodium carbonate as approximately 35 to 36% of the total batch weight;
4. liquid fragrance; i.e. lavender, as approximately 1% to 4% of the total batch weight; and
5. dye or food coloring; e.g blue, as approximately less than 1% of the total batch weight.

The five components may be dry blended together and as feedstock for the second layer in a the tablet press. The appearance of the second layer may be a color other than white, such as blue. Also, the second layer may generate effervescence as water dissolves the citric acid and sodium carbonate components. The amount of effervescence (bubble generation) generated from the second layer may be greater than that generated by the first layer because of the higher percentage by weight of sodium bicarbonate and/or citric acid. Of course, adjusting only the amount citric acid or carbonate salt may also affect the amount of effervescence. Like the first layer, the second layer may liberate a volatile organic fragrance as the lavender component is warmed and released into the air. This middle layer may appear blue.

The composition of the third layer of this example may be a mixture of four components, essentially the same as the first layer.

The shape of this tablet may be a three-layer cylinder such similar to tablet 10, or may have another configuration such as one of those described herein.

### Test Method(s)

The dissolution rate may be determined by the following process:

First a sample unit is made of a test compound intended for use as a single layer in a tablet. The compound ingredients are stirred to a uniform consistency. The compound is shaped into cylindrical discs, 6 mm in thickness and 35 mm in diameter, made by pressing the compound in a tablet press at a pressure of 703 kilogram-force/square centimeter for 5 seconds. The amount of compound used will create discs having a density of about 1.4 grams/cubic centimeters. This amount may be about 6 to 10 grams of compound. The units are equilibrated for 5 days in a room at 20 degrees Centigrade, 30% humidity.

After the equilibration is complete, each unit (preferably a sample size of ten units) is placed in approximately 20 liters of 40 degree Centigrade tap water in a standard stainless steel sink. The depth of the water may be about 10 cm. The time for the product to completely dissolve is determined to be the dissolution time. The dissolution rate is defined as the average dissolution time/per unit. A variation of this method may be used for tablets having multiple layers to determine the overall dissolution rate of a multiple-layer tablet.

The invention disclosed above has been described both in general terms and by reference to specific components and examples. No limitations are intended or imposed by the exemplary materials, combinations and compositions recited, and alternatives will occur to those of ordinary skill in the art without the exercise of inventive faculty. In particular, material identities and weight relationships may be varied from the values set forth above, without departing from the scope of the invention, save as limited by the claims set forth below.

1. A bathing tablet adapted to outwardly exhibit a property change to a user as it dissolves, the bathing tablet comprising:
a first layer;
a second layer disposed on the first layer;
wherein the first layer comprises a different effervescent or exothermic property than the second layer.

2. The bathing tablet of claim 1 wherein the first layer comprises a different visual property than the second layer.

3. The bathing tablet of claim 2 wherein the first layer comprises a different dissolution rate than the second layer.

4. The bathing tablet of claim 1 wherein the first layer comprises a different exothermic property and effervescent property than the second layer.

5. The bathing tablet of claim 1 wherein the first layer comprises a different aromatherapy property than the second layer.

6. The bathing tablet of claim 1 wherein the first layer comprises an effervescent layer and the second layer comprises a non-effervescent material.

7. The bathing tablet of claim 1 wherein the second layer comprises a dummy layer.

8. The bathing tablet of claim 7 wherein the dummy layer comprises indicia on a surface.

9. The bathing tablet of claim 1 further comprising a hidden layer between the first layer and the second layer.

10. The bathing tablet of claim 9 wherein the hidden layer comprises an effervescent material.

11. The bathing tablet of claim 1 further comprising a third layer.

12. The bathing tablet of claim 11 wherein the layers are concentric.

13. The bathing tablet of claim 12 wherein the third layer comprises an effervescent core.

14. The bathing tablet of claim 13 wherein the first layer comprises an effervescent material.

15. A method of manufacturing a multi-layer bathing tablet that will outwardly exhibit a property change to a user as it dissolves, the method comprising the steps of:
preparing an effervescent first compound comprising a binder and a first fragrance;
preparing a second compound comprising a binder and a second fragrance;
layering the first compound on the second compound to create a first layer and a second layer, and pressing the first layer and the second layer together to form a tablet.

16. The method of claim 15 wherein further comprising the steps of preparing a third compound comprising a binder and a third fragrance; and layering and pressing the third layer together with the first layer and second layer to form the tablet.

17. The method of claim 16 wherein the second layer is hidden between the first and third layers.

18. The method of claim 17 wherein the second layer is surrounded by the first layer so that the second layer is hidden.

19. The method of claim 15, wherein the first and second layer are stacked to form a cylinder, wherein all the layers are visible after the pressing the first layer together with the second layer to form a tablet.

20. A bathing tablet adapted to outwardly exhibit a property change to a user as it dissolves, the bathing tablet comprising:
a first layer comprising a binder, first percentage by weight of an acid, a first percentage by weight of a carbonate salt, and a first fragrance;
a second layer disposed on the first layer; the second layer comprising a binder, an second percentage by weight of an acid, a second percentage by weight of a carbonate salt, and a second fragrance.

21. The bathing tablet of claim 20 wherein the first percentage by weight and the second percentage by weight of the acid is unequivalent.

22. The bathing tablet of claim 20 wherein the second percentage by weight and the second percentage by weight of the acid is about equivalent.

23. The bathing tablet of claim 20 wherein the first percentage by weight and the second percentage by weight of the carbonate salt is unequivalent.

24. The bathing tablet of claim 20 wherein the first percentage by weight and the second percentage by weight of the carbonate salt is equivalent.
